(19) United States
(12) Patent Application Publication
(10) Pub. No.: US 2023/0227882 A1
(43) Pub. Date: Jul. 20, 2023

(54) METHODS FOR PRODUCTION OF ANIMAL-FREE HONEY AND MILK SUBSTITUTES

(71) Applicant: Sifotek, AS, Stavanger (NO)

(72) Inventors: Kim Xuyen Phan, Sola (NO); Minh Khanh Nguyen, Winston Salem, NC (US)

(73) Assignee: Sifotek, AS, Stavanger (NO)

(21) Appl. No.: 18/099,450

(22) Filed: Jan. 20, 2023

Related U.S. Application Data
(60) Provisional application No. 63/301,061, filed on Jan. 20, 2022.

(51) Int. Cl. C12P 21/00; A23L 21/25; A23L 33/125; A23L 33/115; A23L 33/175; A23C 11/04; A23C 11/06; A23C 11/10; A23L 2/60; A23L 33/15

(52) U.S. Cl. CPC C12P 21/005; A23L 21/27; A23L 33/125; A23L 33/115; A23L 33/175; A23C 11/04; A23C 11/06; A23C 11/103; A23L 2/60; A23L 33/15

(57) ABSTRACT

Royal jelly honey produced by nurse bees, honey and milk has great values in human health and/or beauty. This invention aims to produce royal jelly honey substitute, honey substitute, animal free milk without the need for bees or cows or goats. First, recombinant major royal jelly proteins (MRJPs), defensin-1, and milk proteins are produced by microbes (such as bacteria, yeasts, or fungi) using precision fermentation. The MRJPs, defensin-1 or milk proteins are then formulated with vitamins, water, minerals, fats, salt and/or amino acids to produce a bee-free royal jelly honey substitute, bee-free honey substitute or animal free milk. MRJPs can also be formulated with vitamins, water, minerals, fats, salt, amino acids and recombinant milk proteins to produce animal-free milk or milk substitutes with supplemental MRJPs. Plant-based milks can also be supplemented with MRJPs and recombinant milk proteins.

The present invention claims priority under 35 USC 119(e) to U.S. Provisional Application No. 63/301,061, filed Jan. 20, 2022, the entire contents of which are incorporated herein by reference.

### FIELD OF THE EMBODIMENTS

The present invention relates a method for producing a royal jelly (RJ) honey substitute, bee-free honey substitute, animal-free milk supplemented with RJ proteins, and a hybrid milk which is a plant-based milk supplemented with recombinant milk proteins and major RJ proteins.

### BACKGROUND OF THE EMBODIMENTS

RJ is a protein-rich mixture of natural products secreted from the gland of worker bees. RJ is considered a functional food and has a wide range of pharmacological activities, including: anti-inflammatory activity, antioxidant activity, antitumor activity, antimicrobial activity, anti-hypercholesterolemic activity, vasodilative activity, anti-lipidemic activity, anti-anemic activity, antibacterial activity, and antifatigue activity, among others. (Kunugi, 2019) (Vezeteu, 2017) (Fan, 2016) (Brudzynski, 2015) (Feng, 2015) (Kashima, 2014) (Majtan, 2010) (Nakaya, 2007) (Kamakura, 2006) (Majtan, 2006) (Simuth, 2004) (Kimura, 2003) (Kamakura, 2001) (Watanabe, 1998) (Watanabe, 1996). In fact, RJ has been widely used to treat health conditions, such as diabetes mellitus, cardiovascular diseases, and cancer (Maleki, 2019) (Pasupuleti, 2017). RJ has also been used as food for health promotion and dietary supplements and as a cosmetic ingredient.

RJ contains 12-15% proteins, 60-70% water, 10-16% sugar, 3-6% fat, and 2-3% vitamins, salts and amino acids. The major protein in RJ are members of the major RJ protein family (MRJPs), which consist of MRJP1, MRJP2, MRJP3, MRJP4, and MRJP5 secreted by worker bees (de Almeida Longuini, 2021) (Tian, 2018). Among them, MRJP1 is the most abundant, with 48% of water-soluble RJ proteins (Tian, 2018) (Simuth, 2001). MRJP3 is reported to strengthen the immune system (Okamoto, 2003).

Despite the great health and beauty benefits of RJ honey, natural production of RJ honey is limited by factors such as decreased bee populations and ethical considerations. Thus, what is needed is a novel method to produce MRJPs and use MRJPs with other components to make a RJ honey substitute.

Also, bee honey, a sweet, viscous food substance, produced by honey bees, includes: sugars (about 64-82.4%), water (about 17.1%), proteins (about 0.1-3.3%), H₂O₂, methyl-glyoxal (MGO), organic acids and phenolic compounds (about 0.2-0.8%), vitamins (about 0.01-0.2%), free amino acids (FAAs), minerals (about 0.04-0.2%), and some volatile compounds. Similar to RJ honey, the quality (e.g., composition, color, aroma, and flavor of honey) depends mainly on the flowers, geographical regions, climates, weather conditions, and bee species.

Further, animal milk (e.g., goat and cow milk) has great nutritional value. Animal milk comprises about 87% water, 5% lactose (carbohydrate), 3.3% fat, 3.3% protein and 0.7% minerals. Milk composition varies depending on the species, like cow, goat or sheep. Generally, milk proteins consist of approximately 82% casein and 18% whey proteins (Davoodi, 2016). The casein protein family includes several types of caseins such as alpha-S1, alpha-S2, beta casein, and kappa casein. Beta casein contains both A1 and A2 beta casein proteins. A1 protein may cause some diseases while A2 does not. Therefore, A2 cow or goat milk is healthier than regular milk which contains both A1 and A2 casein proteins.

Plant-based milk or extracts (such as oat, hemp, hazelnut, sesame, flaxseed, macadamia, soy, almond, coconut, banana, cashew, etc.) are also considered a nutritional substitute for dairy. Plant-based milk is an important alternative for those who are intolerant to lactose components of milk. Non-dairy milk production also has less environmental effects than animal milk production. Moreover, plant-based milk can be supplemented with recombinant milk proteins to increase its protein content. Thus, enhanced methods to create animal-free milk proteins, as well as methods to increase the nutritional value of animal-free milk are also needed.

Examples of related art include:

US20200184381A1 describes methods and systems for engineering and manufacturing collagen-based biomaterials. The methods and systems combine synthetic biology, fermentation, material science and machine learning. Collagen molecules or collagen based materials obtained from using the methods have desired physical or chemical properties such as melting temperature, stiffness, or elasticity. The obtained collagen molecules and sequences are also disclosed.

US20190153068A1 describes non-naturally occurring collagen and elastin molecules. The non-naturally occurring collagens and elastins include truncated collagens, truncated elastins, as well as fusion proteins thereof. The non-naturally occurring collagen and elastin are useful in foods, cosmetics and many other products and uses.

US20180199582A1 describes a method for producing a lactic acid bacteria fermented milk product comprising fermenting milk with both inoculated lactic acid bacteria (LAB) and inoculated Bacillus bacteria.

U.S. Ser. No. 10/595,545B2 describes a substitute ice cream composition comprising: (i) 0.5 weight % to 15 weight % of a recombinant β-lactoglobulin protein comprising a sequence that is at least 90% identical to the bovine protein amino acid sequence, where the recombinant β-lactoglobulin in the substitute ice cream composition confers on the substitute ice cream composition three or more characteristics that are substantially similar to an ice cream produced using bovine milk selected from the group consisting of: taste, appearance, mouthfeel, structure, texture, and emulsification; (ii) ash; and (iii) optionally, one or more lipids. The substitute ice cream composition does not comprise any other milk proteins than the recombinant β-lactoglobulin protein in (i).

CN112236040A describes a polymer comprising a monomeric component of milk protein that has desired properties. In examples, the monomeric component of the milk protein includes a whey protein monomer. In examples, one whey protein monomer is a β-lactoglobulin monomer or an alpha-lactalbumin monomer. In other examples, the monomeric component of the milk protein includes a casein monomer.

US20210037848A1 describes a substitute dairy food composition comprising: (i) a recombinant β-lactoglobulin protein and a β-casein protein; (ii) ash; and (iii) optionally, one or more lipids. The recombinant β-lactoglobulin protein comprises an amino acid sequence that is at least 80% identical to an amino acid sequence of a mammalian β-lactoglobulin protein. The β-casein protein comprises an amino acid sequence that is at least 50% identical to an amino acid sequence of a non-human mammalian β-casein protein. Further, the recombinant β-lactoglobulin protein and the β-casein protein confer on the substitute dairy food composition one or more characteristics of a dairy food product selected from the group consisting of: taste, aroma, appearance, handling, mouthfeel, density, structure, texture, elasticity, springiness, coagulation, binding, leavening, aeration, foaming, creaminess, and emulsification. The substitute dairy food composition does not comprise any other milk proteins other than the recombinant β-lactoglobulin protein and the β-casein protein in (i).

U.S. Ser. No. 11/028,146B2 describes genetically engineered strains of yeast and methods for producing recombinant protein (e.g., collagen). The recombinant protein is used to produce bio-fabricated leather or a material having leather-like properties containing recombinant or engineered collagen. The yeast strains are engineered to produce ascorbate and/or increased production of a ketoglutarate.

WO2021168343A2 describes a recombinant milk protein having an attenuated or essentially eliminated allergenicity, compositions comprising the recombinant milk protein, and methods for producing the recombinant milk and compositions.

U.S. Ser. No. 11/076,615B2 describes methods and compositions including casein, and methods for making these compositions. In a specific example, this reference describes a substitute dairy food composition. The composition includes: (i) a recombinant β-lactoglobulin protein and a κ-casein protein. The recombinant β-lactoglobulin protein comprises an amino acid sequence that is at least 90% identical to an amino acid sequence of a wild-type mammalian β-lactoglobulin protein, and the κ-casein protein comprises an amino acid sequence that is at least 90% identical to an amino acid sequence of a wild-type mammalian κ-casein protein; (ii) ash; and (iii) optionally, one or more lipids. The recombinant β-lactoglobulin protein and the k-casein protein confer on the substitute dairy food composition one or more characteristics of a dairy food product selected from the group consisting of: taste, aroma, appearance, handling, mouthfeel, density, structure, texture, elasticity, springiness, coagulation, binding, leavening, aeration, foaming, creaminess and emulsification and the substitute dairy food composition does not comprise any other milk proteins than the recombinant β-lactoglobulin protein and the κ-casein protein in (i).

WO2020219595A1 describes an egg replacer product. The egg replacer product includes a milk protein component consisting of a subset of whey milk proteins or of a subset of casein milk proteins or of a mixture of a subset of whey milk proteins and a subset of casein milk proteins. The milk protein component imparts or materially contributes to at least one egg attribute of the egg replacer.

US20210037849A1 describes a substitute ice cream composition comprising a recombinant β-lactoglobulin protein and ash. The recombinant β-lactoglobulin protein comprises an amino acid sequence that is at least 80% identical to an amino acid sequence of a mammalian 3-lactoglobulin protein. The substitute ice cream composition does not comprise any other milk proteins than the recombinant β-lactoglobulin protein.

CN102010867A describes a yeast expression method for recombining a major protein AccMRJP1 of apis cerana RJ and product application. CN101669596A describes a RJ honey milk.

CN107495253A describes a RJ honey milk. The RJ honey milk is composed of the following raw materials by weight: 68-89 parts of RJ, 15-30 parts of honey, 5-12 parts of grape seed, 2-10 parts of sucrose, 0.2-1.6 parts of lignin, 0.4-1.2 parts of carotene, 0.3-0.7 part of lysine, 1-5 parts of Adenophora stricta, 3-9 parts of Chinese wolfberry, 1-7 parts of fructus momordicae, 3-9 parts of wrinkled gianthyssop herb, and 1-5 parts of Angelica sinensis.

Some similar systems exist in the art. However, their means of operation are substantially different from the present disclosure, as the other inventions fail to solve all the problems taught by the present disclosure.

### SUMMARY OF THE EMBODIMENTS

The present invention and its embodiments relate to a method for producing a RJ honey substitute, bee-free honey substitute, animal-free milk supplemented with RJ proteins, and a hybrid milk which is a plant-based milk supplemented with recombinant milk proteins and major RJ proteins.

A first embodiment of the present invention describes a method utilizing a yeast expression system to produce a desired recombinant protein. The method includes numerous process steps, such as: utilizing an analyzed protein sequence from a sample of royal jelly honey, bee honey, or animal milk, or utilizing a protein sequence of corresponding royal jelly honey, bee honey or animal milk listed at https://www.ncbi.nlm.nih.gov for plasmid construction. The plasmid constructed encodes for MRJP1, MRJP2, MRJP3, MRJP4, MRJP5, bee defensin-1, alpha-S1, alpha-S2, beta casein, kappa casein, beta-lactoglobulin, alpha-lactalbumin, serum albumin, immunoglobins, lactoferrin, or transferrin. Next, the method includes: inserting the constructed plasmid into microbes via electroporation methods, heat shock or chemical methods, where the microbes may be bacteria, yeast, or fungi.

Then, the method includes culturing the microbes in a stir tank for recombinant protein production, purifying the recombinant proteins, and formulating the recombinant proteins with at least one component to produce a food composition. In examples, the food composition may be a RJ honey substitute, bee-free honey substitute, an animal-free milk, or a hybrid and plant-based milk. The components may include: a lipid, a fat, a free amino acid (FAA), a sugar or a sweetener, a phenolic compound, a vitamin, and/or salt.

The lipid may be sourced or extracted from plants, fruits, or nuts, or may be chemically synthesized. The fat may be salicylic acid, 7-hydroxyoctanoic acid, 3-phenyllactic acid, 8-hydroxyoctanoic acid, 4-hydroxybenzoic acid, 4-hydroxybenzeneacetic acid, 3-hydroxydecanoic acid, 2-octene-1,8-dioic acid, 3,4-dihydroxyphenylethanol, homovanillic acid, 10-hydroxydecanoic acid, protocatechuic acid, 10-hydroxy-2-decenoic acid (10-HAD), sebacic acid, p-coumaric acid, 2-decene-1,10-dioic acid, 3,10-dihydroxydecanoic acid, hexadecenoic acid, ferulic acid, coconut oil, sunflower oil, vegetable oil, and/or grapeseed oil.

The FAA may be aspartic acid (Asp), threonine (Thr), serine (Ser), glutamic acid (Glu), glycine (Gly), alanine (Ala), cysteine (Cys), valine (Val), methionine (Met), isoleucine (Ile), leucine (Leu), tyrosine (Tyr), phenylalanine (Phe), lysine (Lys), histidine (His), arginine (Arg), and/or proline (Pro).

The sugar or sweetener may include one or more sugar compounds, such as: fructose, glucose, sucrose and oligosaccharides. The phenolic compound may be ferulic acid, quercetin, kaempherol, galangin and fisetin, pinocembrin, naringin and hesperidin, apigenin, acacetin, chrysin, or flavonoids, 2-cis, 4-trans abscisic acid, 2-hydroxycinnamic acid, caffeic acid, chlorogenic acid, cinnamic acid, ellagic acid, ferulic acid, gallic acid, p-coumaric acid, 4-hydroxybenzoic acid, protocatechuic acid, sinapic acid, syringic acid, vanillic acid, quercetin, luteolin, pinocembrin, isorhamnetin, kaempferol, chrysin, galangin, pinobanksin and/or 8-methoxy kaempferol.

The vitamin may be vitamin A, vitamin B, vitamin C, vitamin D, vitamin E, or vitamin K. The vitamin B may be thiamine (B1), riboflavin (B2), pyridoxine (B6), pantothenic acid (B5), nicotinic acid, folic acid, biotin, niacin (B3), or cobalamin (B12). The salt may be calcium, CaCl₂), phosphorus, potassium, sodium, KH₂PO₄, citrate, zinc, iron, copper, manganese, trisodium citrate, chloride, barium, nickel, cobalt, lithium, chromium, selenium, arsenic and/or silver.

A second embodiment of the present invention describes a substitute RJ honey composition that includes: one or more MRJPs, one or more phenolic compounds, one or more vitamins, one or more salts, one or more sugars or sweeteners, one or more FAAs, and/or optionally one or more fats.

A third embodiment of the present invention describes a substitute bee-free honey composition that includes: defensin-1 protein, H₂O₂, methyl glyoxal (MGO), one or more phenolic compounds and organic acids, one or more vitamins, one or more salts, one or more sugars or sweeteners, one or more FAAs, and/or optionally one or more fats.

A fourth embodiment of the present invention describes a substitute cow or goat milk composition that includes: one or more casein proteins, one or more whey proteins, one or more vitamins, one or more salts, one or more sugars or sweeteners, and optionally one or more MRJPs, one or more phenolic compounds, one or more fats, and/or one or more FAAs.

A fifth embodiment of the present invention describes a hybrid milk or plant-based milk product supplemented with recombinant proteins that includes: a plant-based milk or extract, one or more milk proteins, one or more vitamins, one or more salts, one or more sugars or sweeteners, and optionally one or more MRJPs, phenolic compounds, fats, and/or FAAs.

A sixth embodiment of the present invention describes an energy shot composition that includes: an amount of bee honey, an amount of a royal jelly honey substitute, and/or an amount of water.

### BRIEF DESCRIPTION OF THE DRAWINGS

FIG. 1 depicts a block diagram associated with method steps involved in a yeast expression system to produce a desired recombinant protein, according to at least some embodiments disclosed herein.

### DESCRIPTION OF THE PREFERRED EMBODIMENTS

The preferred embodiments of the present invention will now be described with reference to the drawings. Identical elements in the various figures are identified with the same reference numerals. Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described herein. The materials, methods, and examples are illustrative only and not intended to be limiting. Other features of the disclosure are apparent from the following detailed description and the claims.

As used herein, the singular forms “a,” “an,” and “the,” are intended to include the plural forms as well, unless the context clearly indicates otherwise.

The phrase “and/or,” as used herein in the specification and in the claims, should be understood to mean “either or both” of the elements so conjoined, i.e., elements that are conjunctively present in some cases and disjunctively present in other cases. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the term “about” is used in conjunction with a numerical range, it modifies that range by extending the boundaries above and below those numerical values. In general, the term “about” is used herein to modify a numerical value above and below the stated value by a variance of 20%, 10%, 5%, or 1%. In certain embodiments, the term “about” is used to modify a numerical value above and below the stated value by a variance of 10%. In certain embodiments, the term “about” is used to modify a numerical value above and below the stated value by a variance of 5%. In certain embodiments, the term “about” is used to modify a numerical value above and below the stated value by a variance of 1%.

When a range of values is listed herein, it is intended to encompass each value and sub-range within that range. For example, “1-5 ng” is intended to encompass 1 ng, 2 ng, 3 ng, 4 ng, 5 ng, 1-2 ng, 1-3 ng, 1-4 ng, 1-5 ng, 2-3 ng, 2-4 ng, 2-5 ng, 3-4 ng, 3-5 ng, and 4-5 ng.

It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

As used herein, the terms “formulation,” “composition,” “food supplement” or “food additive” are used interchangeably throughout the specification and refer to the composition as described herein. The supplement may be in any form, including solid (e.g. a powder), semi-solid (e.g. a food-like consistency/gel), a liquid or alternatively, it may be in the form of a tablet or capsule. The liquid can conveniently be mixed in with the food or ingested directly. The supplement may be high in one or more components of the invention or may be in the form of a combined pack of at least two parts, each part containing the required level of one or more component. In certain embodiments, the disclosed formulation can be in the form of a concentrate that is diluted prior to use. In certain embodiments, the formulation can be supplemented with a pharmaceutical composition.

In general, the present invention describes yeast protein expression systems (e.g., Saccharomyces cerevisiae or Pichia patoris) that are used for the expression and production of recombinant proteins. This technique is a highly developed genetic system, easy to use, and results in reduced time input and reduced costs. Importantly, yeasts are able to carry specifically designed plasmids, and this ability is valuable in a recombinant protein expression system. The plasmid used herein consists of restriction sites that can be used to insert the gene sequence of interest. The transformation of yeasts with this plasmid produces the desired protein and can be appropriately scaled up. In an embodiment, the plasmid used contains a short common sequence, such as 6His, GST or other tags, so it makes purification of the desired protein easy.

As described herein, recombinant RJ, bee honey and milk proteins are synthesized using a precision fermentation method. Specifically, microbes (such as bacteria, yeasts, and fungi, among others not explicitly listed herein) can be genetically engineered to produce proteins, such as: MRJP1, MRJP2, MRJP3, MRJP4, MRJP5, defensin-1, alpha-S1, alpha-S2, beta casein, and kappa casein, beta-lactoglobulin, alpha-lactalbumin, serum albumin, immunoglobins, lactoferrin, and transferrin, among others not explicitly listed herein. These resulting genetically engineered materials can be formulated with other ingredients or components, such as vitamins, amino acid, fats, sugars, minerals, or plant-based milk, to produce nutritional RJ honey or milk substitutes. As MRJPs have many health benefits, their combination with animal-free milk will increase the nutritional value of animal-free milk.

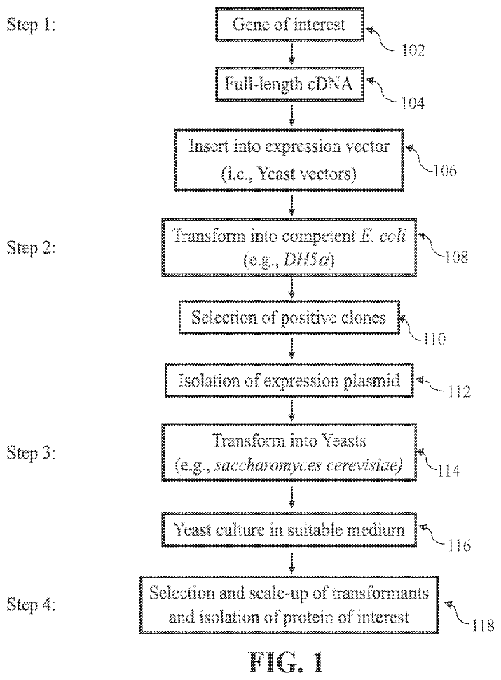

In general, FIG. 1 depicts a block diagram associated with method steps involved in a yeast expression system to produce a desired recombinant protein, according to at least some embodiments disclosed herein. According to the method of FIG. 1, an analyzed protein sequence from a sample of RJ honey or animal milk is used for plasmid construction. The plasmids constructed encodes for MRJP1, MRJP2, MRJP3, MRJP4, MRJP5, defensin-1, alpha-S1, alpha-S2, beta casein, kappa casein, beta-lactoglobulin, alpha-lactalbumin, serum albumin, immunoglobins, lactoferrin, and transferrin. The constructed plasmid can be inserted into microbes (such as bacteria, yeast or fungi) using electroporation or chemical methods. The microbes are then cultured in a stir tank for recombinant protein production. The proteins are purified prior to being formulated with other ingredients or components to produce the nutritional RJ honey or the milk substitute.

Specifically, the method of FIG. 1 includes numerous process steps, such as a Step 1, a Step 2, a Step 3, and a Step 4. The Step 1 includes a process step 102, a process step 104, and a process step 106. The Step 2 includes a process step 108, a process step 110, and a process step 112. The Step 3 includes a process step 114 and a process step 116. The Step 4 includes a process step 118.

### Step 1: Identify Genes of Interest and Plasmid Construction

The method of FIG. 1 begins at the process step 102, which includes identifying a full-length sequence of gene of interest through the National Center for Biotechnology Information (NCBI) search.

Major RJ protein family (MRJPs) and their sequences from Apis mellifera may be obtained based on the NCBI search. The sequences for the Mtn's include the following:
MRJP1: GenBank: AF388203.1 (gene sequence: 1299 bp); Protein ID: AAM73637.1 (protein sequence: 432 aa)
MRJP2: GenBank: AF000632.1 (gene sequence: 1359 bp); Protein ID: AAC61894.1 (protein sequence: 452 aa)
MRJP3: GenBank: NM 001011601.1 (gene sequence: 1635 bp); Protein ID: NP_001011601 (protein sequence: 544 aa)
MRJP4: GenBank: NM 001011610.1 (gene sequence: 1395 bp); Protein ID: NP_001011610.1 (protein sequence: 464 aa)
MRJP5: GenBank: NM 001011599.1 (gene sequence: 1797 bp); Protein ID: NP_001011599.1 (protein sequence: 598 aa)
   The sequences for defensin-1 of Apis mellifera include the following:
Apis mellifera: GenBank: NM 001011616.2 (Gene sequence (CDS): 288 bp) Protein ID: NP_001011616.2 (protein sequence 95 aa)
   The sequences for Casein alpha-S1 (CSN1S1) include the following:
Bos taurus (cow, cattle, bovine): GenBank: NM 181029.2 (gene sequence: 645 bp); Protein ID: NP_851372.1 (protein sequence: 214 aa)
Capra hircus (goat): GenBank: NM 001285695.1 (gene sequence: 642 bp); Protein ID: NP_001272624.1 (protein sequence: 213 aa)
Ovis aries (sheep): GenBank: NM 001009795.1 (Gene sequence: 621 bp); Protein ID: NP_001009795.1 (protein sequence: 206 aa)
   The sequences for Casein alpha-S2 (CSN1S2) include the following:
Bos taurus (cow, cattle, bovine): GenBank: NM 174528.2 (Gene sequence: 669 bp); Protein ID: NP_776953.1 (protein sequence: 222 aa)
Capra hircus (goat): GenBank: NM 001285585.1 (Gene sequence: 672 bp); Protein ID: NP_001272514.1 (protein sequence: 223 aa)
Ovis aries (sheep): GenBank: NM 001009363.1 (Gene sequence: 672 bp); Protein ID: NP_001009363.1 (protein sequence: 223 aa)
   The sequences for beta casein (CSN2) or A2 beta casein include the following:
Bos taurus (cow, cattle, bovine): GenBank: KC993858.1 (Gene sequence: 675 bp); Protein ID: AGT56763.1 (protein sequence: 224 aa)
Capra hircus (goat): GenBank: AH001195.2 (Gene sequence: 669 bp); Protein ID: AAA30906.1 (protein sequence: 222 aa)
Ovis aries (sheep): GenBank: NM 001009373.1 (Gene sequence: 669 bp); Protein ID: NP_001009373.1 (protein sequence: 222 aa)
   The sequences for kappa casein (CSN3) include the following:
Bos taurus (cow, cattle, bovine): GenBank: NM 174294.2 (Gene sequence: 573 bp); Protein ID: NP_776719.1 (protein sequence: 190 aa)
Capra hircus (goat): GenBank: NM 001285587.1 (Gene sequence: 579 bp); Protein ID: NP_001272516.1 (protein sequence: 192 aa)
Ovis aries (sheep): GenBank: NM 001009378.1 (Gene sequence: 579 bp); Protein ID: NP_001009378.1 (protein sequence: 192 aa)
   The sequences for alpha-lactalbumin (LALBA or ALA) include the following:
Bos taurus (cow, cattle, bovine): GenBank: NM 174378.2 (Gene sequence: 429 bp); Protein ID: NP_776803.1 (protein sequence: 142 aa)
Capra hircus (goat): GenBank: NM 001285635.1 (Gene sequence: 429 bp); Protein ID: NP_001272564.1 (protein sequence: 142 aa)
Ovis aries (sheep): GenBank: NM 001009797.1 (Gene sequence: 429 bp); Protein ID: NP_001009797.1 (protein sequence: 142 aa)
   The sequences for beta-lactoglobulin (LGB) include the following:
Bos taurus (cow, cattle, bovine): GenBank: EU883598.1 (Gene sequence: 537 bp); Protein ID: ACG59280.1 (protein sequence: 178 aa)
Capra hircus (goat): GenBank: Z19569.1 (Gene sequence: 543 bp); Protein ID: CAA79623.1 (protein sequence: 180 aa)
Ovis aries (sheep): GenBank: X04520.1 (Gene sequence: 543 bp); Protein ID: CAA28204.1 (protein sequence: 180 aa)
   The sequences for serum albumin (ALB) include the following:
Bos taurus (cow, cattle, bovine): GenBank: NM 180992.2 (Gene sequence: 1824 bp); Protein ID: NP_851335.1 (protein sequence: 607 aa)
Capra hircus (goat): GenBank: XM_005681744.2 (Gene sequence: 1824 bp); Protein ID: XP_005681801.1 (protein sequence: 607 aa)
Ovis aries (sheep): GenBank: NM 001009376.1 (Gene sequence: 1824 bp) Protein ID: NP_001009376.1 (protein sequence: 607 aa)
   The sequences for immunoglobins include the following:
Bos taurus immunoglobin superfamily member 21 (IGSF21), isoform X1: GenBank: XM_024983246.1 (Gene sequence: 1407 bp); Protein ID: XP_024839014.1 (protein sequence: 468 aa)
Bos taurus immunoglobin superfamily member 21 (IGSF21), isoform X2: GenBank: XM_024983250.1 (Gene sequence: 1278 bp); Protein ID: XP_024839018.1 (protein sequence: 425 aa)
Capra hircus immunoglobin superfamily member 21 (IGSF21): GenBank: XM_018054731.1 (Gene sequence: 1407 bp); Protein ID: XP_017910220.1 (protein sequence: 468 aa)
Ovis aries immunoglobin superfamily member 21 (IGSF21): GenBank: XM_027965645.2 (Gene sequence (CDS): 1407 bp); Protein ID: XP_027821446.1 (protein sequence: 468 aa)
   The sequences for lactoferrin (LF) include the following:
Bos taurus (cow, cattle, bovine): GenBank: FJ589071.1 (Gene sequence: 2127 bp); Protein ID: ACM24792.1 (protein sequence: 708 aa)
Capra hircus (goat): GenBank: DQ387456.1 (Gene sequence: 2127 bp) Protein ID: ABD49106.1 (protein sequence: 708 aa)
Ovis aries (sheep): GenBank: AY792499.1 (Gene sequence: 2127 bp); Protein ID: AAV92908.1 (protein sequence: 708 aa)
   The sequences for transferrin or serotransferrin (TF) include the following:
Bos taurus (cow, cattle, bovine): GenBank: NM 177484.3 (Gene sequence: 2115 bp); Protein ID: NP_803450.2 (protein sequence: 704 aa)
Capra hircus (goat): GenBank: XM_005675551.3 (Gene sequence: 2115 bp); Protein ID: XP_005675608.2 (protein sequence: 704 aa)
Ovis aries (sheep): GenBank: XM_042236939.1 (Gene sequence: 3006 bp); Protein ID: XP_042092873.1 (protein sequence: 1001 aa)
   It should be appreciated that these sequences obtained from the NCBI database are incorporated by reference in their entirety.

A plasmid DNA containing this gene sequence (or “insert DNA”) is used as a template for Polymerase Chain Reaction (PCR) amplification. The PCR primers are designed from 5′ and 3′ ends of the gene of interest. Importantly, each primer has a specific restriction site for cloning (usually 6-8 bp), but is not present in the given sequence. The process step 104 follows the process step 102 and includes using the protein gene of interest to create a full-length cDNA. PCR is run to amplify the insert DNA by using a high fidelity Taq DNA polymerase to minimize mutations. When the reaction is done, the PCR product is isolated using the QIAquick PCR Purification Kit.

Here, restriction digestion is performed for the PCR product and destination vector (e.g., yeast vectors) (e.g., the process step 106). The digested DNAs (both the insert DNA and the destination vector) are run on an agarose gel. Then, expected bands are cut and a gel purification is conducted using the QIAquick Gel Extraction Kit.

After gel purification, the concentration of recovered DNAs is determined for ligation. A DNA ligation reaction is conducted to fuse insert DNA to the destination vector. In some examples, 100 ng of total DNA is used in a standard ligation reaction, using T4 DNA ligase reagent (Promega), with an ideal ratio of destination vector to insert DNA being approximately 1:3. The mixture is then incubated at room temperature for about two hours, or at about 16° C. overnight.

Step 2: Transform Expression Plasmid into Competent E. coli for Isolation

The process step 108 follows the process step 106 and includes transforming the expression plasmid into competent E. coli for isolation. The mixture DNA ligation reaction (2 μL) is mixed into 100 μL of competent cells in a microcentrifuge tube and is placed in ice for about 20-30 minutes. The transformation is performed using a heat-shock or electroporation method. For the heat shock method, each transformation tube is carried out by placing the tube into an about 42° C. water bath for about 30-60 seconds. In an embodiment, each transformation tube is carried out by placing the tube into an about 42° C. water bath for about 45 seconds.

For the electroporation method, the transformation is carried out by applying a voltage to the DNA/cell mixture. After heat-shock or electroporation, the tubes are placed back on ice for about 2 minutes and then 1 mL Luria-Bertani (LB) or SOC (Super Optimal broth with Catabolite repression) media is added to the bacteria and growth occurs in an about 37° C. shaking incubator for at least 1 hour. After incubation, some or all of the transformation is plated onto a 10 cm LB agar plate containing the appropriate antibiotic. The plates are incubated at about 37° C. overnight to recover transformants.

Positive clones are selected (e.g., the process step 110) and the final expression plasmid is isolated (e.g., the process step 112). Noticeably, only cells that contain the expression plasmid are able to grow/divide and form colonies in selection marker agar plates. To confirm, about 10 colonies are selected and grown overnight for DNA purification using QIAprep Spin Miniprep Kit. After purifying the DNA, a diagnostic restriction digest is conducted of about 100-300 ng of purified DNA with the enzymes used for the cloning. After enzyme cutting time, the digest is run on an agarose gel to determine the correct clones. Two bands should be visible, one the size of the vector and one the size of the new insert. The plasmid is sequenced to confirm that the final expression plasmid does not have any mutations in its sequence.

Step 3: Transform Isolated Expression Plasmid into Yeasts

The final expression plasmid that carries a selectable marker for screening will be used to transform into a specific yeast strain (e.g., the process step 114). After the transformation, cells are plated on selective media that will only allow transformed cells to grow (e.g., the process step 116). Noticeably, all yeast plasmids carry a normal copy of the yeast URA3 gene, or LYS2, TRP1, MET15, and ura4+ genes, as well as its promoter, so the gene is regulated much like a normal chromosomal gene, and it can be used as a selectable marker for screening.

A yeast deletion strain is used that has the deleted allele of interest (e.g., ura3Δ0). As such, the defective protein, for example Ura3p protein, produced from the plasmid encoded URA3 gene can compensate for the ura3 deletion in the yeast chromosome, allowing transformed cells to grow in the absence of uracil. Because of its reliability, many yeast transformation schemes rely on URA3 complementation to isolate transformants.

The lithium (LiAc) method has been widely used for yeast transformation. This method involves three main steps: (1) preparing competent yeast cells, (2) transformation with plasmid DNA, and (3) subsequent plating to select the transformants. Specifically, yeasts cells are grown aerobically on about 100 mL of yeast extract peptone dextrose (YPD, 1% yeast extract, 2% peptone, and 2% glucose in ddH₂O) medium at about 30° C. with shaking. At the mid-log phase, the cells are harvested by centrifugation, washed once with TE [10 mM tris-HCl (pH 8.0) and 1.0 mM EDTA] and suspended in appropriate volume of TE to get a final concentration of 2×10⁸ cells/mL. The cell suspension (about 0.5 mL) is mixed with an equal volume of about 0.2 M LiAc (lithium acetate), and then incubated with shaking (about 140 rpm) at about 30° C. for about 1 hour. After incubation, about 100 μL of the cell suspension is mixed with about 1 μg of a plasmid DNA and incubated at about 30° C. for about 30 minutes. After incubation, an equal volume of 70% PEG 4000 (Polyethylene glycol 4000) is added and vortexed. After standing for about 1 hour at about 30° C., the suspension mixture is incubated at about 42° C. for about 5 minutes. The cells are cooled down to room temperature and then washed twice with water and resuspended in about 1 mL of water. To select the yeast transformants, about 100 μL of cell suspension is directly spread on synthetic complete (SC) drop-out medium plates. The transformant colonies appear within about 2-4 days at about 30° C.

Alternatively, yeast transformation can be performed by electroporation. Specifically, the yeast cells are grown on about 100 mL of YPD or SD medium supplemented with appropriate nutrients to a density of approximately 1×10⁷ cells/mL at about 30° C. with shaking. Before harvesting, the cultures are placed on ice for about 15 minutes. The cells are collected by centrifugation and washed the resulting pellet thrice with ice-cold sterilized water. The pellet is suspended in ice-cold freezing buffer containing about 0.6-2.5 M sorbitol, about 5-10 mM CaCl₂) and about 10 mM 2-(4-[2-hydroxyethyl]-1-piperazinyl) ethane sulphonic acid (HEPES; pH 7.5) to give a density of approximately about 5×10⁸ cells/mL. Then, about 100 μL of the cell suspension in about 1.5-mL microcentrifuge tubes is frozen and stored in an about −80° C. freezer. For each electroporation, the frozen competent cells are quickly thawed in a water bath at about 30° C. and washed once with about 1 mL of ice-cold 1.0 M sorbitol by centrifugation. The final pellet is suspended in about 1.0 M sorbitol to give a density of about 1×10⁶ cells/mL. The cell suspension is mixed with transforming mix (PEG 4000 950% w/v, LiAc 1M, denatured ssDNA) containing 500 ng of purified plasmid DNA and then is transferred to a chilled cuvette with an about 0.2-cm electrode gap. A 1.5 kV electric pulse is applied to the cell suspension by using the Bio-Rad Gene Pulser Xcell Electroporation System or other corresponding electroporation systems. Immediately, the electroporated cells are diluted in about 1 mL of ice-cold 1.0 M sorbitol, centrifuged at 13,000 rpm for 5 min and the supernatant removed. The cells are washed 3 times with 1 ml deionized water and resuspended in 1 ml drop out media for 1 hour prior to spreading an aliquot (75-100 μL) on minimal selection plates. The transformant colonies appear within about 4-6 days at about 30° C.

### Step 4: Protein Isolation and Purification

The process step 118 follows the process step 116 and includes selection and scale-up of transformants and isolation of the protein of interest. The designed expression plasmid can be strongly replicated in the yeast culture system. The recombinant proteins will be produced as fusion proteins with an N-terminal or C-terminal 6His tag or both. These recombinant proteins are then separated from the yeast and collected by affinity chromatography on a HisTrap nickel column. A complete protocol of recombinant protein purification includes three major steps: (1) a process step 4.1 that includes growth of yeast cells; (2) a process step 4.2 that includes lysis of the yeast cells; and (3) a process step 4.3 that includes purification of the protein.

### Step 4.1: Growth of Yeast Cells

The yeast cells are streaked from a glycerol stock on selective plates and incubated at about 30° C. for about 2-3 days. The yeasts are inoculated as an overnight starter culture in about 3 mL YPD and incubated with shaking at about 30° C. overnight and at about 200 rpm. The yeasts are added to a flask containing 47 ml prewarmed YPD containing about 0.5-2% D-galactose. The cells are then cultured at about 30° C. and about 200 rpm for 3-4 days. The cells are spun down using a high-speed centrifuge with about 500 mL or 1 L capacity bottles at about 5000 rpm, about 4° C. for about 30 minutes and the pellet is resuspended in about 100 mL of an appropriate buffer for breaking open the yeast cells.

### Step 4.2: Lysis of the Yeast Cells

The yeast cell is broken up using a high-pressure homogenizer (HPH). Depending on the type of HPH device used, the procedure will be carried out according to the manufacturer's instructions.

### Step 4.3: Purification of the Protein

The yeast lysate is centrifuged at about 12,000 rpm at about 4° C. for about 30 minutes. A HisTrap nickel column is equilibrated in an appropriate buffer on a fast protein liquid chromatography (FPLC) system. The supernatant is filtered twice: first through a 5 mm filter and then through a 0.8 mm filter. The lysate is loaded onto the nickel column at flow rate of about 2-3 mL per minute. The nickel column is washed with the purification buffer until the UV absorbance at 280 nm reaches the baseline at a flow rate of about 5 mL per minute. The bound protein is eluted with the elution buffer at a flow rate of about 5 mL per minute and the entire eluate is pooled. The eluted protein is concentrated using an Amicon Ultra (Millipore) concentrator to a final volume of about 2 mL.

The sample is applied on a gel filtration column that has been equilibrated in low salt buffer at flow rate of about 0.5 mL per minute and about 1 mL fractions during the run is collected for the purified protein by SDS-PAGE gel and Coomassie staining. Pooled fractions are store on ice at about 4° C. overnight. The sample is placed on a freshly prepared 10 mL phosphor-cellulose column equilibrated in low salt buffer by gravity flow. The column is washed with about 100 mL of low salt buffer. The sample is eluted using a step gradient, four 5 mL fractions of 200 mM KCl buffer followed by four 5 mL fractions of 350 mM KCl buffer, and then the column is washed 1 M KCl buffer.

The different eluted fractions are analyzed by SDS-PAGE gel and Coomassie staining. The fractions are pooled containing stoichiometric amounts of desired protein. The sample protein is dialyzed using a storage buffer containing the His-tag protease to cleave His tag out of desired protein. The protein fractions are concentrated using a centrifugal filter as described to about 0.5−1 mL. The final protein concentration is measured using a spectrophotometer or BCA protein assay. The protein is stored at about −80° C.

As explained, the protein of interest generated from the method of FIG. 1 may be incorporated into a food product, such as a RJ honey substitute, an animal-free milk supplemented with RJ proteins, or a hybrid and plant-based milk supplemented with recombinant milk proteins and major RJ proteins, among others not explicitly listed herein. The food product may include one or more ingredients or components, such as: lipids or fats, free amino acids (FAAs), sugars or sweeteners, phenolic compounds, vitamins, and/or salts, among other components or ingredients not explicitly listed herein.

The lipids or fats may include one or more lipids or fats. The lipids can be sourced or extracted from plants, fruits, nuts, or can also be chemically synthesized. Non-limiting examples of fats include: salicylic acid, 7-hydroxyoctanoic acid, 3-phenyllactic acid, 8-hydroxyoctanoic acid, 4-hydroxybenzoic acid, 4-hydroxybenzeneacetic acid, 3-hydroxydecanoic acid, 2-octene-1,8-dioic acid, 3,4-dihydroxyphenylethanol, homovanillic acid, 10-hydroxydecanoic acid, protocatechuic acid, 10-hydroxy-2-decenoic acid (10-HAD), sebacic acid, p-coumaric acid, 2-decene-1,10-dioic acid, 3,10-dihydroxydecanoic acid, hexadecenoic acid, and/or ferulic acid, among others not explicitly listed herein. Among these, 10-HAD is the major lipid component in RJ that has many pharmacological properties, which is an important lipid to determine the quality of RJ.

The FAAs may have one or more FAAs. Non-limiting examples of FAAs include aspartic acid (Asp), threonine (Thr), serine (Ser), glutamic acid (Glu), glycine (Gly), alanine (Ala), cysteine (Cys), valine (Val), methionine (Met), isoleucine (Ile), leucine (Leu), tyrosine (Tyr), phenylalanine (Phe), lysine (Lys), histidine (His), arginine (Arg), and/or proline (Pro), among others not explicitly listed herein.

The sugars or sweeteners may include one or more sugar compounds, such as fructose, glucose and/or oligosaccharides, among others not explicitly listed herein.

The phenolic compounds may include one or more phenolic compound. Non-limiting examples of phenolic compounds include: ferulic acid, quercetin, kaempherol, galangin and fisetin, pinocembrin, naringin and hesperidin, apigenin, acacetin, chrysin, and/or flavonoids, among others not explicitly listed herein.

The vitamins may include one or more vitamins, such as vitamin A, B, C, D, E, and/or K, among others not explicitly listed herein. Non-limiting examples of the vitamins include, for example, thiamine (B1), riboflavin (B2), pyridoxine (B6), pantothenic acid (B5), nicotinic acid, folic acid, biotin, niacin (B3), and/or cobalamin (B12), among others not explicitly listed herein.

The salt may include one or more salts. Non-limiting examples of salts include: calcium, CaCl₂), phosphorus, potassium, sodium, KH₂PO₄, citrate, zinc, iron, copper, manganese, trisodium citrate, and/or chloride, among others not explicitly listed herein.

### EXAMPLES

### Example 1

Example 1 produces a RJ honey substitute. In this example, recombinant MRJPs (purchased from Mybiosourse.com, MRJP1 Cat #MBS1134959, MRJP2 Cat #MBS1128312, MRJP3 Cat #MBS1296531, MRJP4 Cat #MBS1473814, MRJP5 Cat #MBS1113840) were formulated with other components to make the RJ honey alternative. The other components included: water, sugar, fats, salts, vitamins, and free amino acids. All water and sugar, fat, salts and vitamins were purchased from Sigma Aldrich. For example, fatty acids such as 10-hydroxy-2-decenoic acid (10-HAD, Cat #SML3381) and 10-hydroxydecanoic acid (Cat #379700) were purchased from Sigma Aldrich. Water, sugar, fats, salts, vitamins, and free amino acids were mixed with a mechanical homogenizer (VWR® 250 Homogenizer), then the proteins were added to the mix. The composition of recombinant MRJPs ranges from about 0-90 wt. % of the total volume of the final RJ honeys. Water ranges from about 0-95 wt. %, the sugar content ranges from about 0.01-30 wt. %, the fat content ranges from about 0.01-30 wt. %, the vitamin content ranges from about 0.01-50 wt. %, the amino acid content ranges from about 0.01-50 wt. %, and the salt content ranges from about 0.0001-5 wt. % of the total volume of the final RJ honeys.

### Example 2

Example 2 produces an animal free milk (also called a synthetic milk or a milk alternative) with a RJ honey supplement. Calcium phosphate and vitamins were homogenized with alpha-S1 alpha-S2, beta casein at a speed of 1000 rpm, then the pH was adjusted to 6.0. Kapa casein was added under mixing. After that, beta-lactoglobulin, alpha-lactalbumin, MRJP1 were added under mixing. The composition of recombinant casein proteins (alpha-S1, alpha-S2, beta casein, kappa casein) ranges from about 0-50 wt. %, the composition of recombinant whey proteins (beta-lactoglobulin, alpha-lactalbumin) ranges from about 0-50 wt. %, the composition of recombinant MRJPs ranges from about 0-50 wt. %, the amount of water ranges from about 0-95 wt. %, the sugar content ranges from about 0-30 wt. %, the fat content ranges from about 0-30 wt. %, the vitamin content ranges from about 0-50 wt. %, the amino acid content ranges from about 0-50 wt. %, and the salt content ranges from about 0-5 wt. % of the total volume of the final milk substitute.

### Example 3

Example 3 produces a milk powder or milk protein powder. The milk in examples 1 and 2 were formulated into a milk powder using a spray dryer B290 (Buchi) at 80° C. Each milk protein powder was also produced using a spray dryer B290 (Buchi) at 70-120° C.

### Example 4

Example 4 produces a plant-based milk with recombinant milk proteins or RJ honey proteins (also called a hybrid milk). Plant-based milk or extracts from plants, fruits, or nuts (such as oat, hemp, hazelnut, sesame, flaxseed, macadamia, soy, almond, coconut, banana, or cashew, etc.) can also be formulated with recombinant MRJPs or recombinant milk proteins (e.g., casein and whey) to produce the recombinant protein-rich plant-based milk or hybrid milk. Specifically, Oatly oak milk was purchased from a local grocery store, beta lactoglobulin was added to the milk. MRJP1 was also added to the milk. The mixture was mixed until complete dissolution of the whey and MRJP proteins. The composition of the plant-based milk or extracts ranges from about 0-98 wt. %, the amount of water ranges from about 0-95 wt. %, the composition of MRJPs ranges from about 0-50 wt. %, the composition of casein proteins ranges from about 0-50 wt. %, and the composition of whey proteins ranges from about 0-50 wt. % of the total volume of the hybrid milk.

### Example 5

Example 5 produces an energy shot that includes bee honey supplemented with RJ. Bee honey was purchased from a local grocery store, and was mixed with the RJ honey substitute in Example 1. The composition of the bee honey ranges from about 1-50 wt. %, the composition of RJ honey substitute ranges from about 1-20 wt. %, and an amount of water ranges from about 0-90% of the total volume of the energy shot.

In an embodiment, the present invention relates to systems and methods of utilizing a yeast expression system to produce one or more recombinant proteins, the method comprising:
utilizing an analyzed protein sequence from a sample of royal jelly honey, bee honey, or animal milk to generate a constructed plasmid;
inserting the constructed plasmid into one or more microbes;
culturing the one or more microbes in a stir tank to generate one or more recombinant proteins;
purifying the one or more recombinant proteins; and
formulating the one or more recombinant proteins with at least one component to produce a food composition.

In a variation, the constructed plasmid encodes for MRJP1, MRJP2, MRJP3, MRJP4, MRJP5, defensin-1, alpha-S1, alpha-S2, beta casein, kappa casein, beta-lactoglobulin, alpha-lactalbumin, serum albumin, immunoglobins, lactoferrin, and/or transferrin.

In a variation, the one or more microbes are one or more members selected from the group consisting of bacteria, yeast, fungi, and algae.

In an embodiment, the inserting the constructed plasmid into the one or more microbes occurs via one or more of an electroporation method, heat shock or a chemical method.

In a variation, the food composition comprises a RJ honey substitute, a bee-free honey substitute, an animal-free milk, or a hybrid milk.

In a variation, the at least one component comprises one or more of: a lipid, a fat, an oil, a free amino acid (FAA), a sugar, a sweetener, a phenolic compound, a vitamin, or a salt.

In a variation, the lipid is sourced or extracted from plants, fruits, or nuts, or is generated by microbe fermentation or is chemically synthesized.

In a variation, the fat and/or the oil is one or more members selected from the group consisting of: salicylic acid, 7-hydroxyoctanoic acid, 3-phenyllactic acid, 8-hydroxyoctanoic acid, 4-hydroxybenzoic acid, 4-hydroxybenzeneacetic acid, 3-hydroxydecanoic acid, 2-octene-1,8-dioic acid, 3,4-dihydroxyphenylethanol, homovanillic acid, 10-hydroxydecanoic acid, protocatechuic acid, 10-hydroxy-2-decenoic acid (10-HAD), sebacic acid, p-coumaric acid, 2-decene-1,10-dioic acid, 3,10-dihydroxydecanoic acid, hexadecenoic acid, ferulic acid, grape seed oil, sunflower oil, soy bean oi, vegetable oil, and coconut oil.

In a variation, the FAA is one or more members selected from the group consisting of: aspartic acid (Asp), threonine (Thr), serine (Ser), glutamic acid (Glu), glycine (Gly), alanine (Ala), cysteine (Cys), valine (Val), methionine (Met), isoleucine (Ile), leucine (Leu), tyrosine (Tyr), phenylalanine (Phe), lysine (Lys), histidine (His), arginine (Arg), and proline (Pro).

In a variation of the system or method, the sugar or sweetener comprises one or more sugar compounds, and wherein the one or more sugar compounds are one or more members selected from the group consisting of: fructose, dextrose, sucrose, glucose, oligosaccharides, stevia, erythritol, and monk fruit extract.

In a variation, the phenolic compound is one or more members selected from the group consisting of: ferulic acid, quercetin, kaempherol, galangin, fisetin, pinocembrin, naringin, hesperidin, apigenin, acacetin, chrysin, and flavonoids.

In a variation, the vitamin is one or more members selected from the group consisting of: vitamin A, vitamin B, vitamin C, vitamin D, vitamin E, and vitamin K.

In a variation, the vitamin is vitamin B, and wherein the vitamin B is one or more members selected from the group consisting of: thiamine (B1), riboflavin (B2), pyridoxine (B6), pantothenic acid (B5), nicotinic acid, folic acid, biotin, niacin (B3), and cobalamin (B12).

In a variation, the salt is one or more members selected from the group consisting of: calcium, CaCl₂), phosphorus, potassium, sodium, KH₂PO₄, citrate, zinc, iron, copper, manganese, trisodium citrate, and chloride.

In an embodiment, the present invention relates to a substitute royal jelly (RJ) honey composition comprising:
one or more major RJ proteins (MRJPs);
one or more phenolic compounds;
one or more vitamins;
one or more salts;
one or more sugars or sweeteners;
one or more fats or oils; and
one or more free amino acids (FAAs).

In a variation, the composition comprises one or more fats and/or oils and they are one or more members selected from the group consisting of: salicylic acid, 7-hydroxyoctanoic acid, 3-phenyllactic acid, 8-hydroxyoctanoic acid, 4-hydroxybenzoic acid, 4-hydroxybenzeneacetic acid, 3-hydroxydecanoic acid, 2-octene-1,8-dioic acid, 3,4-dihydroxyphenylethanol, homovanillic acid, 10-hydroxydecanoic acid, protocatechuic acid, 10-hydroxy-2-decenoic acid (10-HAD), sebacic acid, p-coumaric acid, 2-decene-1,10-dioic acid, 3,10-dihydroxydecanoic acid, hexadecenoic acid, ferulic acid, 2-cis, 4-trans abscisic acid, 2-hydroxycinnamic acid, caffeic acid, chlorogenic acid, cinnamic acid, ellagic acid, ferulic acid, gallic acid, p-coumaric acid, 4-hydroxybenzoic acid, protocatechuic acid, sinapic acid, syringic acid, vanillic acid, quercetin, luteolin, pinocembrin, isorhamnetin, kaempferol, chrysin, galangin, pinobanksin and 8-methoxy kaempferol.

In a variation, the one or more MRJP's are one or more members selected from the group consisting of: MRJP1, MRJP2, MRJP3, MRJP4, MRJP5, MRJP6, MRJP7, MRJP8, and MRJP9.

In a variation, the one or more phenolic compounds are one or more members selected from the group consisting of: ferulic acid, quercetin, kaempherol, galangin, fisetin, pinocembrin, naringin, hesperidin, apigenin, acacetin, chrysin, and flavonoids.

In a variation, the one or more vitamins are one or more members selected from the group consisting of: vitamin A, vitamin B, vitamin C, vitamin D, vitamin E, and vitamin K.

In a variation, the one or more salts are one or more members selected from the group consisting of: calcium, CaCl₂), phosphorus, potassium, sodium, KH₂PO₄, citrate, zinc, iron, copper, manganese, trisodium citrate, and chloride.

In a variation of the composition, the one or more sugars or sweeteners comprise one or more sugar compounds, and wherein the one or more sugar compounds are one or more members selected from the group consisting of: fructose, sucrose, dextrose, glucose, oligosaccharides, stevia, erythritol, and monk fruit extract.

In a variation, the one or more FAAs are one or more members selected from the group consisting of: aspartic acid (Asp), threonine (Thr), serine (Ser), glutamic acid (Glu), glycine (Gly), alanine (Ala), cysteine (Cys), valine (Val), methionine (Met), isoleucine (Ile), leucine (Leu), tyrosine (Tyr), phenylalanine (Phe), lysine (Lys), histidine (His), arginine (Arg), and proline (Pro).

In an embodiment, the present invention relates to a bee-free honey composition comprising:
a defensin-1 protein;
hydrogen peroxide (H₂O₂);
methyl-glyoxal (MGO);
one or more organic acids and/or phenolic compounds;
one or more vitamins;
one or more salts; and
one or more sugars and/or sweeteners.

In a variation, the composition further comprises:
one or more free amino acids (FAAs).

In a variation, the one or more FAAs are one or more members selected from the group consisting of: aspartic acid (Asp), threonine (Thr), serine (Ser), glutamic acid (Glu), glycine (Gly), alanine (Ala), cysteine (Cys), valine (Val), methionine (Met), isoleucine (Ile), leucine (Leu), tyrosine (Tyr), phenylalanine (Phe), lysine (Lys), histidine (His), arginine (Arg), and proline (Pro).

In a variation, the one or more sugars and/or sweeteners comprise one or more sugar compounds, and
the one or more sugar compounds are one or more members selected from the group consisting of: fructose, sucrose, dextrose, glucose and oligosaccharides.

In a variation of the composition, the one or more organic acids and/or phenolic compounds are one or more members selected from the group consisting of: 2-cis, 4-trans abscisic acid, 2-hydroxycinnamic acid, caffeic acid, chlorogenic acid, cinnamic acid, ellagic acid, ferulic acid, gallic acid, p-coumaric acid, 4-hydroxybenzoic acid, protocatechuic acid, sinapic acid, syringic acid, vanillic acid, quercetin, luteolin, pinocembrin, isorhamnetin, kaempferol, chrysin, galangin, pinobanksin and 8-methoxy kaempferol.

In a variation, the one or more vitamins are one or more members selected from the group consisting of: vitamin A, vitamin B, vitamin C, vitamin D, vitamin E, and vitamin K.

In a variation, the one or more salts are one or more members selected from the group consisting of: calcium, CaCl₂), phosphorus, potassium, sodium, KH₂PO₄, citrate, zinc, iron, copper, manganese, trisodium citrate, chloride, barium, nickel, cobalt, lithium, chromium, selenium, arsenic and silver.

In an embodiment, the present invention relates to a substitute cow or goat milk composition comprising:
two or more casein proteins;
one or more whey proteins;
one or more vitamins;
one or more salts;
one or more fats and/or oils;
one or more flavoring agents; and
one or more sugars and/or sweeteners.

In a variation, the composition further comprises:
one or more RJ proteins.

In a variation, the composition further comprises one or more phenolic compounds, wherein the one or more phenolic compounds are one or more members selected from the group consisting of: ferulic acid, quercetin, kaempherol, galangin, fisetin, pinocembrin, naringin, hesperidin, apigenin, acacetin, chrysin, and flavonoids.

In a variation, the one or more vitamins are one or more members selected from the group consisting of: vitamin A, vitamin B, vitamin C, vitamin D, vitamin E, and vitamin K.

In a variation, the one or more salts are one or more members selected from the group consisting of: calcium, CaCl₂), phosphorus, potassium, sodium, KH₂PO₄, citrate, zinc, iron, copper, manganese, trisodium citrate, chloride, barium, nickel, cobalt, lithium, chromium, selenium, arsenic and silver.

In a variation of the composition, the one or more sugars or sweeteners comprise one or more sugar compounds, and the one or more sugar compounds are one or more members selected from the group consisting of: fructose, glucose, sucrose, dextrose, and oligosaccharides.

In a variation the composition further comprises:
two or more major casein proteins, wherein the two or more major casein proteins are two or more members selected from the group consisting of: alpha-S1, alpha-S2, beta casein, kappa casein, and/or
one or more whey proteins, where the one or more whey proteins are one or more members selected from the group consisting of beta-lactoglobulin, alpha-lactalbumin, serum albumin, immunoglobins, lactoferrin, and transferrin.

In a variation of the composition, the composition further comprises:
one or more major RJ proteins (MRJPs), wherein the one or more MRJPs are one or more members selected from the group consisting of: MRJP1, MRJP2, MRJP3, MRJP4, and MRJP5.

In a variation, the one or more fats and/or oils are selected from the group consisting of: salicylic acid, 7-hydroxyoctanoic acid, 3-phenyllactic acid, 8-hydroxyoctanoic acid, 4-hydroxybenzoic acid, 4-hydroxybenzeneacetic acid, 3-hydroxydecanoic acid, 2-octene-1,8-dioic acid, 3,4-dihydroxyphenylethanol, homovanillic acid, 10-hydroxydecanoic acid, protocatechuic acid, 10-hydroxy-2-decenoic acid (10-HAD), sebacic acid, p-coumaric acid, 2-decene-1,10-dioic acid, 3,10-dihydroxydecanoic acid, hexadecenoic acid, ferulic acid, grape seed oil, sunflower oil, peanut oil, vegetable oil, and coconut oil.

In a variation of the composition, the composition further comprises:
one or more free amino acids (FAAs), wherein the one or more FAAs are one or more members selected from the group consisting of: aspartic acid (Asp), threonine (Thr), serine (Ser), glutamic acid (Glu), glycine (Gly), alanine (Ala), cysteine (Cys), valine (Val), methionine (Met), isoleucine (Ile), leucine (Leu), tyrosine (Tyr), phenylalanine (Phe), lysine (Lys), histidine (His), arginine (Arg), and proline (Pro).

In an embodiment, the present invention relates to a substitute A2 cow or goat milk composition comprising:
an A2 beta casein protein;
one or more casein proteins other than an A2 beta casein protein;
one or more whey proteins;
optionally, one or more RJ proteins;
one or more vitamins;
one or more salts;
one or more fats and/or oils;
one or more flavoring agents; and
one or more sugars and/or sweeteners.

In a variation, the composition further comprises:
one or more RJ proteins.

In a variation of the composition, the composition further comprises
one or more phenolic compounds, wherein the one or more phenolic compounds are one or more members selected from the group consisting of: ferulic acid, quercetin, kaempherol, galangin, fisetin, pinocembrin, naringin, hesperidin, apigenin, acacetin, chrysin, and flavonoids.

In a variation, the one or more vitamins are one or more members selected from the group consisting of: vitamin A, vitamin B, vitamin C, vitamin D, vitamin E, and vitamin K.

In a variation, the one or more salts are one or more members selected from the group consisting of: calcium, CaCl₂), phosphorus, potassium, sodium, KH₂PO₄, citrate, zinc, iron, copper, manganese, trisodium citrate, chloride, barium, nickel, cobalt, lithium, chromium, selenium, arsenic and silver.

In a variation, the one or more sugars or sweeteners comprise one or more sugar compounds, and wherein the one or more sugar compounds are one or more members selected from the group consisting of: fructose, glucose, sucrose, dextrose, and oligosaccharides.

In an embodiment, the composition further comprises:
A2 beta casein,
   and/or one or more major casein proteins, wherein the one or more major casein proteins are one or more members selected from the group consisting of: alpha-S1, alpha-S2, and kappa casein, and/or one or more whey proteins, wherein the one or more whey proteins are one or more members selected from the group consisting of beta-lactoglobulin, alpha-lactalbumin, serum albumin, immunoglobins, lactoferrin, and transferrin.

In a variation, the composition further comprises:
one or more major RJ proteins (MRJPs), wherein the one or more MRJPs are one or more members selected from the group consisting of: MRJP1, MRJP2, MRJP3, MRJP4, and MRJP5.

In a variation, the one or more fats and/or oils are one or more members selected from the group consisting of: salicylic acid, 7-hydroxyoctanoic acid, 3-phenyllactic acid, 8-hydroxyoctanoic acid, 4-hydroxybenzoic acid, 4-hydroxybenzeneacetic acid, 3-hydroxydecanoic acid, 2-octene-1,8-dioic acid, 3,4-dihydroxyphenylethanol, homovanillic acid, 10-hydroxydecanoic acid, protocatechuic acid, 10-hydroxy-2-decenoic acid (10-HAD), sebacic acid, p-coumaric acid, 2-decene-1,10-dioic acid, 3,10-dihydroxydecanoic acid, hexadecenoic acid, ferulic acid, grape seed oil, sunflower oil, peanut oil, vegetable oil, and coconut oil.

In a variation, the composition further comprises:
one or more free amino acids (FAAs), wherein the one or more FAAs are one or more members selected from the group consisting of: aspartic acid (Asp), threonine (Thr), serine (Ser), glutamic acid (Glu), glycine (Gly), alanine (Ala), cysteine (Cys), valine (Val), methionine (Met), isoleucine (Ile), leucine (Leu), tyrosine (Tyr), phenylalanine (Phe), lysine (Lys), histidine (His), arginine (Arg), and proline (Pro).

In an embodiment, the present invention relates to a hybrid milk or plant-based milk product supplemented with recombinant proteins, the hybrid milk or plant-based milk product comprising:
a plant-based milk or extract;
one or more recombinant milk proteins;
one or more vitamins;
one or more salts;
one or more fats and/or oils; and
one or more sugars and/or sweeteners.

In a variation, the hybrid milk or the plant-based milk product further comprises:
one or more major milk proteins, wherein the one or more major milk proteins are one or more members selected from the group consisting of: alpha-S1, alpha-S2, beta casein, kappa casein, beta-lactoglobulin, alpha-lactalbumin, serum albumin, immunoglobins, lactoferrin, and transferrin.

In a variation, the hybrid milk or the plant-based milk product further comprises:
one or more phenolic compounds, wherein the one or more phenolic compounds are one or more members selected from the group consisting of: ferulic acid, quercetin, kaempherol, galangin, fisetin, pinocembrin, naringin, hesperidin, apigenin, acacetin, chrysin, and flavonoids.

In a variation, the one or more fats and/or oils are one or more members selected from the group consisting of: salicylic acid, 7-hydroxyoctanoic acid, 3-phenyllactic acid, 8-hydroxyoctanoic acid, 4-hydroxybenzoic acid, 4-hydroxybenzeneacetic acid, 3-hydroxydecanoic acid, 2-octene-1,8-dioic acid, 3,4-dihydroxyphenylethanol, homovanillic acid, 10-hydroxydecanoic acid, protocatechuic acid, 10-hydroxy-2-decenoic acid (10-HAD), sebacic acid, p-coumaric acid, 2-decene-1,10-dioic acid, 3,10-dihydroxydecanoic acid, hexadecenoic acid, ferulic acid, grape seed oil, sunflower oil, peanut oil, vegetable oil, and coconut oil.

In a variation, the hybrid milk or the plant-based milk product further comprises:
one or more free amino acids (FAAs), wherein the one or more FAAs are one or more members selected from the group consisting of: aspartic acid (Asp), threonine (Thr), serine (Ser), glutamic acid (Glu), glycine (Gly), alanine (Ala), cysteine (Cys), valine (Val), methionine (Met), isoleucine (Ile), leucine (Leu), tyrosine (Tyr), phenylalanine (Phe), lysine (Lys), histidine (His), arginine (Arg), and proline (Pro).

In an embodiment, the present invention relates to a milk powder product comprising:
calcium phosphate,
one or more vitamins
alpha-S1, alpha-S2, beta casein,
Kapa casein
beta-lactoglobulin,
alpha-lactalbumin, and
one or more MRJPs.

In a variation, any of the compositions described herein can be dried into a powder via spray drying, freeze drying, shelf drying, using a bed dryer, drum/roller drying, supercritical drying or dielectric drying.

In a variation, the present invention relates to a powder of a single protein, wherein the powder comprises one or more members selected from the group consisting of alpha-S1, alpha-S2, beta casein, kappa casein, beta-lactoglobulin, alpha-lactalbumin, serum albumin, immunoglobins, lactoferrin, transferrin, and MRJPs.

In an embodiment, the present invention relates to proteins selected from the group consisting of alpha-S1, alpha-S2, beta casein, kappa casein, beta-lactoglobulin, alpha-lactalbumin, serum albumin, immunoglobins, lactoferrin, transferrin, and MRJPs.

In a variation, the protein can be dried into a powder via spray drying, freeze drying, shelf drying, using a bed dryer, drum/roller drying, supercritical drying or dielectric drying.

In a variation, the present invention relates to an energy shot composition comprising:
bee or bee-free honey;
a royal jelly honey substitute; and
water.

The energy shot composition may have any of the metabolites/compounds that are present in any of the compositions that are disclosed herein.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others or ordinary skill in the art to understand the embodiments disclosed herein.

Although this invention has been described with a certain degree of particularity, it is to be understood that the present disclosure has been made only by way of illustration and that numerous changes in the details of construction and arrangement of parts may be resorted to without departing from the spirit and the scope of the invention.

### REFERENCES

The following references, to the extent that they provide exemplary procedural or other details supplementary to those set forth herein or for other purposes, are specifically incorporated herein by reference.
Brudzynski, K., et al., MRJP1-Containing Glycoproteins Isolated from Honey, a Novel Antibacterial Drug Candidate With Broad Spectrum Activity Against Multi-Drug Resistant Clinical Isolates, Front. Microbiol., 2015, Vol. 6, Issue 711, DOI: 10.3389/fmicb.2015.00711.
Davoodi S. H., et al., Health-Related Aspects of Milk Proteins. Iran J Pharm Res., 2016, Vol. 15, Issue 3, Pages 573-591, PMID: 27980594.
De Almeida Longuini A., et al., Supplementation with an Inorganic Zinc Source in the Metalloproteomic Profile of Royal Jelly in Apis mellifera L., Biol Trace Elem Res., 2021, Vol. 199, Issue 11, Pages 4308-4318, DOI: 10.1007/s12011-020-02564-3.
Fan, P., et al., Functional and Proteomic Investigations Reveal Major Royal Jelly Protein 1 Associated with Anti-Hypertension Activity in Mouse Vascular Smooth Muscle Cells, Sci. Rep., 2016, Vol. 6, Page 30230.
Feng, M., et al., In-Depth N-Glycosylation Reveals Species-Specific Modifications and Functions of the Royal Jelly Protein from Western (Apis mellifera) and Eastern Honeybees (apis cerana), J. Proteome Res., 2015, Vol. 14, Pages 5327-5340, DOI: 10.1021/acs.jproteome.5b00829.
Kamakura, M. & Sakaki, T. A Hypopharyngeal Gland Protein of the Worker Honeybee Apis mellifera L. Enhances Proliferation of Primary-Cultured Rat Hepatocytes and Suppresses Apoptosis in the Absence Of Serum, Protein Expr. Purif., 2006, Vol. 45, Pages 307-314, DOI: 10.1016/j.pep 0.2005.08.004.
Kamakura, M., et al., Fifty-Seven-Kda Protein in Royal Jelly Enhances Proliferation of Primary Cultured Rat Hepatocytes and Increases Albumin Production in the Absence of Serum, Biochem. Biophys. Res. Commun., 2001, Vol. 282, Pages 865-874, DOI: 10.1006/bbrc.2001.4656.
Kashima, Y., et al., Identification of a Novel Hypocholesterolemic Protein, Major Royal Jelly Protein 1, Derived From Royal Jelly, PloS ONE, 2014, Vol. 9, Page e105073, DOI: 10.1371/journal.pone.0105073.
Kimura, M., et al., 350-Kda Royal Jelly Glycoprotein (Apisin), Which Stimulates Proliferation of Human Monocytes, Bears the B1-3Galactosylated N-Glycan-Analysis of the N-Glycosylation Site, Biosci. Biotech. Biochem., 2003, Vol. 67, Pages 2055-2058, DOI: 10.1271/bbb.67.2055.
Kunugi H., et al., Royal Jelly and Its Components Promote Healthy Aging and Longevity: From Animal Models to Humans. Int J Mol Sci., 2019, Vol. 20, Issue 19, DOI: 10.3390/ijms20194662.
Majtan, J., et al., Effect of Honey and its Major Royal Jelly Protein 1 on Cytokine and MMP-9 MRNA Transcripts in Human Keratinocytes. Exp. Dermatol. 2010, Vol. 19, Pages e73-e79, DOI: 10.1111/j.1600-0625.2009.00994.x.
Majtan, J., et al., The Immunostimulatory Effect of the Recombinant Apalbumin 1-Major Honeybee Royal Jelly Protein-On TNF-alpha Release, Int. Immunopharmacol., 2006, Vol. 6, Pages 269-278, DOI: 10.1016/j.intimp.2005.08.014.
Maleki, V., et al., Effects of Royal Jelly on Metabolic Variables in Diabetes Mellitus: A Systematic Review, Complement. Ther. Med., 2019, Vol. 43, Pages 20-27, DOI: 10.1016/j.ctim.2018.12.022.
Nakaya, M., et al., Effect of Royal Jelly On Bisphenol A-Induced Proliferation of Human Breast Cancer Cells, Biosci. Biotechnol. Biochem., 2007, Vol. 71, Pages 253-255, DOI: 10.1271/bbb.60453.
Okamoto, I., et al., Major Royal Jelly Protein 3 Modulates Immune Responses in vitro and in vivo, Life Sciences, 2003, Vol. 73, Pages 2029-45, DOI: 10.1016/s0024-3205(03)00562-9.
Pasupuleti V. R., et al., Honey, Propolis, and Royal Jelly: A Comprehensive Review of Their Biological Actions and Health Benefits. Oxid. Med. Cell Longev., 2017, DOI: 10.1155/2017/1259510.
Simuth, J., et al., Immunochemical Approach to Detection of Adulteration in Honey: Physiologically Active Royal Jelly Protein Stimulating TNF-Alpha Release is a Regular Component of Honey, J. Agric. Food Chem., 2004, Vol. 52, Pages 2154-2158, DOI: 10.1021/jf034777y.
Simuth, J. Some Properties of the Main Protein of Honeybee (Apis Mellifera) Royal Jelly, Apidologie, 2001, Vol. 32, Pages 69-80, DOI: 10.1051/apido:2001112.
Tian W., et al., Architecture of the Native Major Royal Jelly Protein 1 Oligomer, Nat Commun., 2018, Vol. 9, Issue 1, DOI:10.1038/s41467-018-05619-1.
Vezeteu, T. V., et al., Food to Some, Poison to Others—Honeybee Royal Jelly and its Growth Inhibiting Effect on European Foulbrood Bacteria, Microbiologyopen, 2017, Vol. 6, Page e00397, DOI: 10.1002/mbo3.397.
Watanabe, K., et al., Stimulation of Cell Growth in the U-937 Human Myeloid Cell Line by Honey Royal Jelly Protein, Cytotechnology, 1998, Vol. 26, Pages 23-27, DOI: 10.1023/A:1007928408128.
Watanabe, K., et al., Growth Stimulation with Honey Royal Jelly DIII Protein of Human Lymphocytic Cell Lines in a Serum-Free Medium, Biotechnol. Tech., 1996, Vol. 10, Pages 959-962, DOI: 10.1007/bf00180402.

What is claimed is:

1. A method utilizing a yeast expression system to produce one or more recombinant proteins, the method comprising:
utilizing an analyzed protein sequence from a sample of royal jelly honey, bee honey, or animal milk to generate a constructed plasmid;
inserting the constructed plasmid into one or more microbes;
culturing the one or more microbes in a stir tank to generate one or more recombinant proteins;
purifying the one or more recombinant proteins; and
formulating the one or more recombinant proteins with at least one component to produce a food composition.

2. The method of claim 1, wherein the constructed plasmid encodes for MRJP1, MRJP2, MRJP3, MRJP4, MRJP5, defensin-1, alpha-S1, alpha-S2, beta casein, kappa casein, beta-lactoglobulin, alpha-lactalbumin, serum albumin, immunoglobins, lactoferrin, and/or transferrin.

3. The method of claim 1, wherein the one or more microbes are selected from the group consisting of bacteria, yeast, fungi, and algae.

4. The method of claim 1, wherein the inserting the constructed plasmid into the one or more microbes occurs via one or more of an electroporation method, heat shock or a chemical method.

5. The method of claim 1, wherein the food composition comprises a RJ honey substitute, a bee-free honey substitute, an animal-free milk, milk powder, or a hybrid milk.

6. A substitute royal jelly (RJ) honey composition comprising:
one or more major RJ proteins (MRJPs);
one or more phenolic compounds;
one or more vitamins;
one or more salts;
one or more sugars or sweeteners;
one or more fats or oils; and
one or more free amino acids (FAAs).

7. The substitute RJ honey composition of claim 6, wherein each of the one or more fats or oils are one or more members selected from the group consisting of: salicylic acid, 7-hydroxyoctanoic acid, 3-phenyllactic acid, 8-hydroxyoctanoic acid, 4-hydroxybenzoic acid, 4-hydroxybenzeneacetic acid, 3-hydroxydecanoic acid, 2-octene-1,8-dioic acid, 3,4-dihydroxyphenylethanol, homovanillic acid, 10-hydroxydecanoic acid, protocatechuic acid, 10-hydroxy-2-decenoic acid (10-HAD), sebacic acid, p-coumaric acid, 2-decene-1,10-dioic acid, 3,10-dihydroxydecanoic acid, hexadecenoic acid, ferulic acid, 2-cis, 4-trans abscisic acid, 2-hydroxycinnamic acid, caffeic acid, chlorogenic acid, cinnamic acid, ellagic acid, ferulic acid, gallic acid, p-coumaric acid, 4-hydroxybenzoic acid, protocatechuic acid, sinapic acid, syringic acid, vanillic acid, quercetin, luteolin, pinocembrin, isorhamnetin, kaempferol, chrysin, galangin, pinobanksin and 8-methoxy kaempferol.

8. The substitute RJ honey composition of claim 6, wherein each of the one or more MRJPs are one or more members selected from the group consisting of: MRJP1, MRJP2, MRJP3, MRJP4, MRJP5, MRJP6, MRJP7, MRJP8, and MRJP9.

9. A bee-free honey composition comprising:
a defensin-1 protein;
hydrogen peroxide (H₂O₂);
methyl-glyoxal (MGO);
one or more organic acids and/or phenolic compounds;
one or more vitamins;
one or more salts; and
one or more sugars and/or sweeteners.

10. A substitute cow or goat milk composition comprising:
two or more casein proteins;
one or more whey proteins;
one or more vitamins;
one or more salts;
one or more fats and/or oils;
one or more flavoring agents; and
one or more sugars and/or sweeteners.

11. The substitute cow or goat milk composition of claim 10, further comprising:
one or more RJ proteins.

12. The substitute cow or goat milk composition of claim 10, further comprising
one or more phenolic compounds, wherein the one or more phenolic compounds are one or more members selected from the group consisting of: ferulic acid, quercetin, kaempherol, galangin, fisetin, pinocembrin, naringin, hesperidin, apigenin, acacetin, chrysin, and flavonoids.

13. The substitute cow or goat milk composition of claim 10, wherein the one or more vitamins are one or more members selected from the group consisting of: vitamin A, vitamin B, vitamin C, vitamin D, vitamin E, and vitamin K.

14. The substitute cow or goat milk composition of claim 10, wherein the one or more salts are one or more members selected from the group consisting of: calcium, CaCl₂), phosphorus, potassium, sodium, KH₂PO₄, citrate, zinc, iron, copper, manganese, trisodium citrate, chloride, barium, nickel, cobalt, lithium, chromium, selenium, arsenic and silver.

15. The substitute cow or goat milk composition of claim 10,
wherein the one or more sugars or sweeteners comprise one or more sugar compounds, and
wherein the one or more sugar compounds are one or more members selected from the group consisting of: fructose, glucose, sucrose, dextrose, and oligosaccharides.

16. The substitute cow or goat milk composition of claim 10, further comprising:
two or more major casein proteins, wherein each of the two or more major casein proteins are two or more members selected from the group consisting of: alpha-S1, alpha-S2, beta casein, kappa casein, and/or
one or more whey proteins, where the one or more whey proteins are one or more members selected from the group consisting of beta-lactoglobulin, alpha-lactalbumin, serum albumin, immunoglobins, lactoferrin, and transferrin.

17. The substitute cow or goat milk composition of claim 10, further comprising:
one or more major RJ proteins (MRJPs), wherein the one or more MRJPs are one or more members selected from the group consisting of: MRJP1, MRJP2, MRJP3, MRJP4, and MRJP5.

18. The substitute cow or goat milk composition of claim 10, wherein the one or more fats or oils are selected from the group consisting of: salicylic acid, 7-hydroxyoctanoic acid, 3-phenyllactic acid, 8-hydroxyoctanoic acid, 4-hydroxybenzoic acid, 4-hydroxybenzeneacetic acid, 3-hydroxydecanoic acid, 2-octene-1,8-dioic acid, 3,4-dihydroxyphenylethanol, homovanillic acid, 10-hydroxydecanoic acid, protocatechuic acid, 10-hydroxy-2-decenoic acid (10-HAD), sebacic acid, p-coumaric acid, 2-decene-1,10-dioic acid, 3,10-dihydroxydecanoic acid, hexadecanoic acid, hexadecenoic acid, ferulic acid, grape seed oil, sunflower oil, peanut oil, vegetable oil, and coconut oil.

19. The substitute cow or goat milk composition of claim 10, further comprising:
one or more free amino acids (FAAs), wherein the one or more FAAs are one or more members selected from the group consisting of: aspartic acid (Asp), threonine (Thr), serine (Ser), glutamic acid (Glu), glycine (Gly), alanine (Ala), cysteine (Cys), valine (Val), methionine (Met), isoleucine (Ile), leucine (Leu), tyrosine (Tyr), phenylalanine (Phe), lysine (Lys), histidine (His), arginine (Arg), and proline (Pro).

20. A substitute A2 cow or goat milk of claim 19 composition further comprising:
an A2 beta casein protein;
one or more casein proteins other than an A2 beta casein protein;
one or more whey proteins;
optionally, one or more RJ proteins;
one or more vitamins;
one or more salts;
one or more fats and/or oils;
one or more flavoring agents; and
one or more sugars and/or sweeteners.

21. The substitute A2 cow or goat milk composition of claim 20, further comprising:
one or more RJ proteins.

22. The recombinant proteins of claim 1, further comprising a hybrid milk or plant-based milk product comprising:
a plant-based milk or extract;
one or more recombinant milk proteins;
one or more vitamins;
one or more salts;
one or more fats and/or oils; and
one or more sugars and/or sweeteners.

23. The hybrid milk or the plant-based milk product of claim 22, further comprising:
one or more major milk proteins, wherein the one or more major milk proteins are one or more members selected from the group consisting of: alpha-S1, alpha-S2, beta casein, kappa casein, beta-lactoglobulin, alpha-lactalbumin, serum albumin, immunoglobins, lactoferrin, and transferrin.

24. A milk powder product of claim 5 comprising:
calcium phosphate,
one or more vitamins
alpha-S1, alpha-S2, beta casein,
Kapa casein
beta-lactoglobulin,
alpha-lactalbumin, and
one or more MRJPs.

25. The substitute A2 cow or goat milk composition of claim 20, wherein the composition is dried into a powder via spray drying, freeze drying, shelf drying, using a bed dryer, drum/roller drying, supercritical drying or dielectric drying.

26. The hybrid milk or plant-based milk product of claim 22, wherein the hybrid milk or plant-based milk product is dried into powder via spray drying, freeze drying, shelf drying, using a bed dryer, drum/roller drying, supercritical drying or dielectric drying.

27. The powder of claim 26 further comprising a single protein, wherein the powder comprises one or more members selected from the group consisting of alpha-S1, alpha-S2, beta casein, kappa casein, beta-lactoglobulin, alpha-lactalbumin, serum albumin, immunoglobins, lactoferrin, transferrin, and MRJPs.

28. The one or more proteins according to claim 27 selected from the group consisting of alpha-S1, alpha-S2, beta casein, kappa casein, beta-lactoglobulin, alpha-lactalbumin, serum albumin, immunoglobins, lactoferrin, transferrin, and MRJPs.

29. The powder of the single protein of claim 27, wherein the powder is dried into powder via spray drying, freeze drying, shelf drying, using a bed dryer, drum/roller drying, supercritical drying or dielectric drying.

30. A royal jelly honey substitute composition according to claim 6 further comprising:
bee or bee-free honey;
an energy shot; and
water.
